(19) United States
(12) Patent Application Publication
(10) Pub. No.: US 2022/0218618 A1
(43) Pub. Date: Jul. 14, 2022

(54) DOSING SYSTEM FOR CONTROLLED SUBSTANCES RELEASE BY MEANS OF DOSING MEMBRANES

(71) Applicant: Anna POROPAT, Buje (HR)

(72) Inventor: Anna POROPAT, Buje (HR)

(21) Appl. No.: 17/595,352

(22) PCT Filed: Apr. 28, 2020
(86) PCT No.: PCT/HR2020/000004

§ 371 Date: Nov. 15, 2021

(30) Foreign Application Priority Data
May 16, 2019 (HR) P20190905A

(51) Int. Cl. A61K 9/48; A61J 3/07

(52) U.S. Cl. CPC A61K 9/4808; A61J 3/071

(57) ABSTRACT

The membrane dosing system for controlled release of substances from the container provides controlled release characteristics (delayed, prolonged and intensity) of one or more active substances from one or more different parts of the same container housed in a medium that dissolves (erodes) membranes into an environment. The dosage system shown consists of three basic parts, two outer elements (2 and 4) and one internal dividing element (3) which is connecting the outer elements (2 and 4) and forming two compartments (5 and 6). The membranes (7 and 8) can be of different shapes and thicknesses. Compartments (5 and 6) are spaces for inserting a substance that may be same or different. The thickness, diameter and shape of the membranes (7 and 8) determine the moment at which the contents will start to leak from of one individual compartment of the (capsule) container into the environment.

### TECHNICAL FIELD TO WHICH THE INVENTION RELATES

This invention relates to a dosing system of membranes on container walls which allow controlled release characteristics (delayed, sustained and intensity) of one or more active substances from one or more of the same or different compartments of the same container in the environment.

### TECHNICAL PROBLEM

Some existing methods are not suitable for controlled, and in some cases personalized features of substance release to influence the targeted processes. The invention solves the stated with a membrane construction of the container whose content can be one or more of the same and/or different substances. The problem is solved by different thicknesses of membranes and detailed construction designs of the membranes at specific locations of the container wall.

### THE STATE OF THE ART

Despite the advanced technology, it still in some cases does not offer appropriate and simple solutions.

A particularly characteristic example is in medicine and the pharmaceutical industry in the administration of drugs to humans and animals. Conventional oral dosage forms are mainly formulated so that almost all the amount of active substance is released shortly after ingestion. Such commercial dosage forms do not sufficiently take care of controlling the release rate of the substance after the dosage form has been introduced into the organism. The result is fast release of large proportion of the dose of the substance which is larger than it is required, thus reducing the efficacy of the drug. There are dosage forms of the drug that allow modifying the release profile, but their disadvantage is the demanding manufacture in terms of the use of combination of suitable materials and construction of the embodiment. In the case of programmed substance release, such release profile (time-released substance ammount dependency) improves therapeutic efficacy and user compatibility, which is not the case with the conventional solutions.

A similar application of the dosage of active substances may be found in other branches of activity. The invention is particularly destined for areas where the mentioned cannot be carried out by other suitable methods. A particular application of the invention is possible in the food industry, defense industry and other chemical industry.

### SUMMARY OF THE INVENTION

The primary objective is to provide controlled release characteristics (delayed, prolonged, continuous and intensity) of one or more substances from one or more different parts of the same container into the environment.

The secondary objective is that the invention is inexpensive and easy to manufacture and provides efficient application (filling, stacking and releasing the substance in the medium). In order to achieve the desired release profil, it is necessary that the container is housed in a suitable medium that dissolves (erodes) the walls of the container and thus releases the substance from the container. The membranes may be part of the walls or part of the fixtures that meet the required characteristics.

The invention is a system of one or more of the same and/or different dosing membranes (dispensers) on container wall while the container may have one or more compartments for one or more different substances.

Membranes (dosing systems) may be located on the container walls or parts of individual compartments. The dosing system consists of a membrane, and in principle it is a thinned wall on which the dosing system is located. Hole-covering membrane may be of different geometric shapes and dimensions according to needs and desired release profile. The thickness of the membrane is also adapted to the specific needs. In order to achieve the desired controlled release mechanism, it is necessary to dissolve the membrane faster or before the other walls of the container, whether this is achieved by the thickness, dimensions, shape and/or type of material from which the membrane will be made. Special attention is paid to the construction of parts of the membrane, such as:

connection of the membrane to the wall of the container in the form of slopes and/or radii membrane position (outer part of the tank wall, inside the thickness of the tank wall, inner wall of the tank).

All dimensions are determined in accordance with predefined needs because they depend on several assumptions (the type of material of which ail the elements of the container are made, including the dosing system of the membrane) and on the expected results in application according to the primary object of the invention.

### BRIEF DESCRIPTION OF DRAWINGS

The accompanying drawings, which are included in the description and which make the description of the invention, illustrate the before said problems:

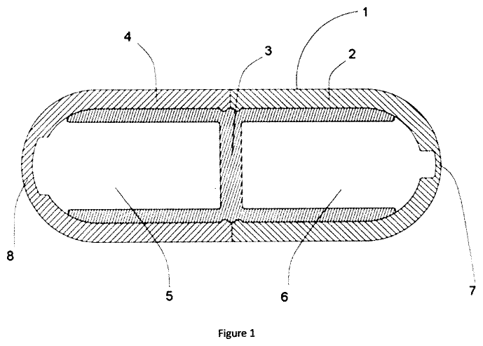

1. FIG. 1—the illustration of dosing membrane capsules for oral use in the cross-section

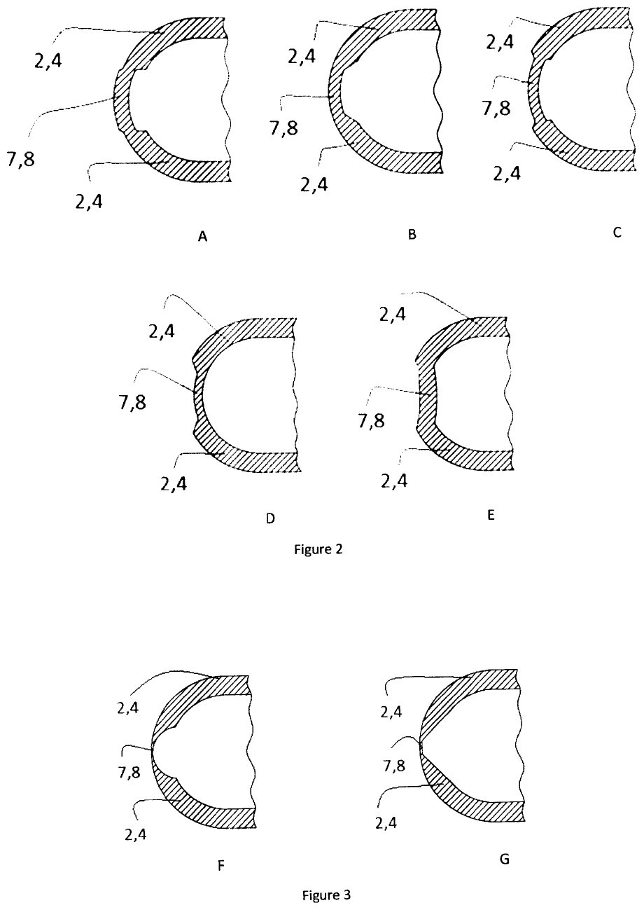

2. FIG. 2—the illustration of the position and details of the dosing membrane connection in relation to the thickness of the tank wall

3. FIG. 3—the illustration of a dosing membrane with uneven thickness

### A DETAILED DESCRIPTION OF AT LEAST ONE OF THE EMBODIMENTS OF THE INVENTION

As can be seen from the drawings in the appendix, the application of dosing membranes is shown in the example of a capsule.

FIG. 1 shows a cylindrical capsule with spherical ends in cross section.

The shown capsule consists of three basic parts, two outer elements 2 and 4 and one inner dividing element 3 that connects the outer elements 2 and 4 and makes two compartments 5 and 6. The membranes 7 and 8 can be of different shapes. In the case of a circular shape, the thickness of the membrane is from 0.5 mm to 99% of the thickness of the walls of the outer elements, and the diameter of the circle of the membrane is from 1 mm to the size of the diameter of the capsule cylinder. The thickness of the membrane is the same throughout the surface of the membrane and less than the thickness of the walls. The thickness of the inner dividing element 3, which is also the divider of sections 5 and 6, has a thickness that is defined according to the desired effects. Sections 5 and 6 of the container insert a substance that may be the same or different. The thickness of the membranes 7 and 8 determines the moment at which the contents from one capsule container will release. When administered orally to the body, in the event that the aim is to endure the complete container without releasing the substance into the stomach and enter the intestines, the thinner membrane 7 is releasing the substance from the interior of compartment 6 first into the intestines and after the estimated release time from compartment 6, the release begins from the compartment 5 through the membrane 8. Due to the proper decomposition of all the walls, discharge takes place through the cavity 8 from compartment 5 of the capsule. By the described system, it is shown that the container has endured without leakage of the substance in the stomach medium. In the intestines a programmed prolonged release is achieved which can be combined with the impulse effect by affecting the different performances of the dosing membranes of the individual parts of the capsule. On the other hand, if the goal is releasing in the stomach, the thickness and material are determined according to the conditions in the stomach that will affect the degradation of the walls. By combining the thickness of the membranes (or the type of material of the membrane), a suitable combination of delayed and prolonged release of the active substances in the capsule can be achieved.

FIG. 2 shows examples of the position and details of the connection of the dosing membranes relative to the thickness of the container walls.

The positions of the dosing membranes relative to the wall are shown as follows:

A—the dosing membrane is on the outside of the thickness of the container wall

B—the dosing membrane is at the level of outside the thickness of the container wall

C—the dosing membrane is within the wall thickness of the container wall

D—the dosing membrane is at the level of the inner thickness of the container wall

E—the dosing membrane is on the inside of the thickness of the container walls

FIG. 2 also shows some forms of connections of the membrane and the tank wall, with a sharp joint or a slight joint (slopes and radii). Different variations are possible according to needs. The membranes can be made of the same material as the tank walls but also of materials of different characteristics.

Instead of the shown compound connection of the dosing membrane and the wall, the dosing membrane can be made as a separate element of suitable characteristics, which is inserted into the corresponding opening on the container wall. The surfaces of the dosing membranes can be of different regular or irregular geometric shapes according to the needs.

FIG. 3 shows examples of dosing membrane of uneven thickness.

In order to achieve corresponding effects, dosage membranes of unequal thickness may be used. The picture shows:

F—dosing membrane in the form of sphere and

G—dosing membrane in the form of a truncated cone or pyramid. Other shapes are also possible such as concave or convex lenses, double truncated cones or pyramids and similar.

The choice of the membrane position with respect to the outer element wall, the geometry of the connection/joint of the membrane and the outer element wall, and the geometry of the shape and thickness of the membrane is dependent on the desired target, taking also into account the swelling effect of the material which the membrane (and the capsule wall) is made of.

The mentioned is particularly noteworthy when PVA (polyvinyl alcohol) material is used, which is the most common case.

### Method of Application of the Invention

The primary objective is to provide controlled release characteristics (delayed, prolonged, continuous and intensity) of one or more substances from one or more of the same or different parts of the same dosing system into the environment in a cheap, simple and effective manner.

### LIST OF USED REFERENCE SIGNS

1—section of capsule

2—external element

3—internal dividing element

4—external element

5—section

6—section

7—thinner membrane

8—thicker membrane

1-4. (canceled)

5. Dosing system for the release of an active substance comprising one or more membranes (7, 8) and one or more exterior container elements (2, 4), wherein an active substance release profile, preferably a prolonged or delayed release profile, is defined by the membranes' geometrical shape.

6. Container according to claim 5, wherein the membranes (7,8) are connected to the exterior container elements (2, 4) with sharp or slight joints.

7. Container according to claim 5, wherein the geometrical shape is a sphere, truncated cone, double truncated cone, pyramid, cylinder, concave lense or convex lense.

8. Container according to claim 5, wherein at least one of the shapes is truncated.

9. Container according to claim 5, wherein the membrane is bent towards the capsule interior.

10. Container according to claim 5, wherein the membrane protrudes from the container.

11. Container according to claim 5, wherein the container is at least partially composed of polyvinyl alcohol.
